(19) United States
(12) Patent Application Publication
(10) Pub. No.: US 2013/0331870 A1
(43) Pub. Date: Dec. 12, 2013

(54) PRESSURIZATION OPTIMIZATION F0R CORNEAL GRAFT PREPARATION

(71) Applicant: Tissue Banks International, Baltimore, MD (US)

(72) Inventor: Jonathan Glynn HARGIS, Memphis, TN (US)

(21) Appl. No.: 13/915,987

(22) Filed: Jun. 12, 2013

Related U.S. Application Data
(60) Provisional application No. 61/658,460, filed on Jun. 12, 2012.

(51) Int. Cl. A61F 9/013

(52) U.S. Cl. CPC A61F 9/0133
USPC 606/166

(57) ABSTRACT

Described herein are methods for accurate and precise preparation of corneal grafts. The utilization of pressure optimization in preparing corneal grafts allows for increased accuracy in regards to the residual posterior bed. The increased accuracy allows for greater precision to meet the surgeon's preferences. The greater precision allows for residual posterior bed thickness of grafts that can provide better visual acuity results in recipients.

### CROSS-REFERENCE TO RELATED APPLICATION

This application claims priority to U.S. Provisional Application for Patent Ser. No. 61/658,460 filed Jun. 12, 2012, the contents of which are incorporated herein by reference in their entirety.

### TECHNICAL FIELD

The invention relates to methods for increasing accuracy and precision in thicknesses of corneal grafts.

### BACKGROUND

The cornea represents the anterior portion of the eye shell and is free of blood vessels. Because of its transparency and curvature, the cornea behaves as a powerful positive lens that focuses light on the central part of the retina. The preservation of corneal transparency and physiological curvature is essential for vision.

The cornea includes three different types of tissue lying adherently on one another, i.e., (from the outside to the inside), epithelium, stroma, and endothelium. The epithelium include several layers of cells kept together very tightly by means of so-called tight-junctions and serves the main purpose of preventing any foreign material or substance or microorganism from penetrating into the deeper layers (protective function).

The stroma builds the skeleton of the cornea, of which represents more than 90% in thickness. It is made of organized collagen fibrils arranged in layers. The direction of the fibres in each layer is orthogonal to the direction of the fibres in the overlying and the underlying layers. In general, the distance between the fibrils is lower than half of the wavelength of visible light, and this characteristic results in the transparency of the cornea. If the distance increases, e.g., when water enters the cornea and causes edema, the transparency goes lost.

The more internal layer, is a monolayer of specialized flat cells that lines the posterior surface of the cornea and faces the anterior chamber of the eye, being in contact with the aqueous humor. This monolayer is called endothelium and sits on a basal membrane, which it produces itself, called Descemet's membrane. The main function of the endothelium is to keep the hydration of the cornea to a relatively low level (about 70%). This is possible because of the presence of a bicarbonate pump in the endothelial cells, which continuously removes from the cornea the fluid that naturally enters it from the anterior chamber. The mechanism by which said removal is achieved, known as endothelial pump, includes carriers and ion channels promoting the flux of ions from the stroma to the aqueous humor (mainly Na⁺ and HCO₃⁻) followed by a water movement.

The human corneal endothelium does not show mitotic activity and the number of its cells slowly decreases with age (about 0.5% per year starting from adulthood). Moreover, various ocular diseases can speed the loss of corneal endothelial cells. Any endothelial disease resulting in a strong reduction of endothelial cell density also reduces the endothelial pump function. When this reaches a level that is not sufficient to eliminate all the water naturally entering the cornea, the cornea itself becomes thicker (edematous) and looses transparency.

All diseases causing a serious alteration of corneal curvature and/or thickness can result in partial or complete blindness. If diseases cannot be cured with therapeutic compositions, the damage they produce is irreversible and corneal transplant represents the sole therapeutic option.

The diseases leading to corneal curvature alterations are represented by keratoconus which is a congenital ectasia (i.e., a condition of inner eye pressure pushing out against a thinned corneal wall, causing it to bulge resulting in worsening vision over time) affecting young individuals and causing a corneal deformation into a more conical shape than its normal gradual curve resulting in a substantial distortion of vision. Transparency alterations comprise congenital diseases, corneal dystrophy, inherited diseases damaging the corneal endothelium or causing an accumulation of anomalous substances in the corneal stroma with consequences on vision that become more severe during adulthood; inflammatory diseases such as the infection produced by Herpes virus; degenerative diseases such as bullous kerathopathy resulting from failure of the corneal endothelium to maintain the normally dehydrated state of the cornea that can be caused by corneal endothelial trauma and can occur during intraocular surgery (e.g., cataract removal) or after placement of a poorly designed or malpositioned intraocular lens implant, promoting the development of bullous keratopathy; traumatic injuries such as perforations. Finally, a further group is represented by subjects undergoing a further transplantation because of a rejection or because of the exhaustion of the corneal flap effectiveness. About 50% of corneal transplants are performed because of corneal endothelium disorders.

### Corneal Transplant

Corneal transplant involves the substitution of the central corneal region with a homologous corneal flap having, normally, a diameter of 8.0-8.5 mm (penetrating keratoplasty, PK). The surgery is performed using corneas explanted from selected donors in total or local anaesthesia and lasts between 30 and 60 minutes. The healthy tissue is sutured at the residual portion of the pathological cornea with a nylon thread 10/0 that is hence left in situ for about 12 to 18 months. The procedure requires few days of hospitalization or can be performed outpatient surgery; it is followed by a relatively slow visual recovery and usually does not require systemic immunosuppressive therapy. Rejection is limited because the cornea does not contain blood vessels. If a rejection occurs, it can often be controlled by topical and systemic steroidal therapy.

Lamellar keratoplasty (LK) substitutes only the superficial pathologic layers of an altered cornea hence leaving the deepest, healthy, corneal structures untouched. Depending on the surgical technique, the LK can be either superficial (substitution of the anterior part of corneal stroma), deep (substitution of the whole corneal stroma down to the Descemet's membrane) posterior (substitution of the posterior part of the corneal stroma together with the Descemet's membrane and the corneal endothelium).

In 2002, deep lamellar technique was developed in order to separate the corneal stroma from the underlying Descemet membrane; it is referred to as the “big bubble technique.” Anwar and Teichmann, J. Cataract Refract. Surg. 28(3): 398-403 (2002). The procedure, performed on the patient's eye, involves generating a big air bubble between the stroma and the Descemet's membrane. Removal of the stroma exposes the smooth Descemet's membrane. The technique requires the use of a keratoplasty 16 blade ring marker on the cornea, a partial trephination (300 microns) with pre-set depth followed by the insertion of a bent 27 G needle attached to an air filled syringe down into the corneal groove the needle being advanced deep into the paracentral stroma at about 80% depth. Once the needle is correctly in place, the plunger is pressed with some force in order to form a bubble recognizable by a white circle that allows removal of the stroma anterior to the bubble with a blade. A side entry is created peripherally to the bubble allowing some aqueous to exit from the eye. The bubble is penetrated with a sharp blade)(˜30°) and the knife is withdrawn letting the bubble collapse. A specific spatula is inserted into the cavity of the bubble through the opening created by the sharp blade; the stroma above the spatula is sliced with the blade and the residual stroma is removed with specific scissors. Finally, the Descemet's membrane is stripped off the donor button, the donor stroma and epithelium is sutured into place with 10/0 nylon sutures and the tension adjusted using a keratoscope.

The “big bubble” technique was developed for patients with good endothelium and served the purpose of transplanting all but the endothelium from a donor cornea into a recipient eye.

However, another way to separate Descemet's membrane and endothelium from the rest of the cornea by means of an air injection can be used to perform a different type of surgery, aimed at replacing only the diseased endothelium and Descemet's membrane from the affected cornea with healthy donor endothelium and Descemet. In these patients, the extreme thinness of the tissue to be excised makes the preparation of the donor graft extremely difficult with the techniques described and employed to date.

Posterior LK is used for substituting endothelium and Descemet's membrane, while leaving intact the still healthy anterior corneal layers. The posterior lamellar keratoplasty (e.g., either Deep Lamellar Endothelial Keratoplasty (DLEK) or Descemet's Stripping Endothelial Keratoplasty (DSEK)) provides several advantages over PK for the treatment of corneal diseases because only the tissue in need of transplantation is removed and the healthy tissue is left intact. Further, the suturing drawbacks of PK are avoided.

In DLEK, a manual deep stromal dissection is carried out to remove a thin posterior stromal lamella together with the underlying endothelium. The procedure is therefore technically challenging with high risk of perforation and most of the entire surface obtained with manual dissection is quite rough and of poor optical quality, thus negatively affecting the final visual result.

DSEK is technically easier, because the stromal dissection of the recipient cornea is substituted by simple peeling of Descemet's membrane and endothelium from the posterior surface of the recipient cornea. However, DSEK still requires manual dissection for the preparation of the donor lamella. Waste of donor tissue because of perforation while preparing the graft and the persistence of an interface with a hand-dissected surface are the major disadvantages of this procedure.

A further evolution of DSEK is DSAEK (Descemet Stripping Automated Endothelial Keratoplasty): the only difference being the preparation of the donor graft, which in DSAEK is dissected by means of a microkeratome that provides a perfectly smooth surface on the donor side of the corneal interface, thus allowing faster visual rehabilitation and better final visual acuity.

In 2006, Melles described the newest technique of endothelial keratoplasty, the so called “Descemet's Membrane Endothelial Keratoplasty (DMEK).” Melles et al., Cornea 25(8): 987-990 (2006). In this procedure, the donor graft consists solely of the Descemet's membrane and the underlying endothelium, which is placed on the posterior surface of the recipient cornea after removal of the diseased endothelium and Descemet. This procedure avoids the formation of a stromal interface, which may negatively affect vision when present. In addition to the skills required to perform the other types of endothelial keratoplasty, here the surgeon is confronted with the difficult task of removing from the donor cornea the endothelial monolayer together with Descemet's membrane, avoiding any possible damage to the tissue to be transplanted. Preparation of the donor tissue for DMEK is highly dependent on the surgeon's surgical skills. Further, handling the “ultra-thin” donor tissue while inserting it into the eye and placing into proper position requires difficult manipulation and positioning of the donor tissue.

Preparation of the Donor Endothelial/Descemet's Graft

The primary method described to separate donor Descemet's membrane together with the endothelium form the overlying stroma employs manual stripping using specially designed micro-instruments (spatula, hook, etc.). The corneal endothelium is a monolayer of cells lying on its basal membrane (Descemet's membrane) and strictly adherent onto the stroma posterior corneal surface. The technique developed by Dr. Melles and described in US 2005/0010244, requires a tool described in the same application that allows the removal of the Descemet's membrane and the corneal endothelium attached thereto, together with the lower part of the corneal stroma. In this technique, a donor button (i.e., lenticule) is incised in the area of the scleral spur and a superficial dissection is performed to separate the posterior corneal layers (thin layer of posterior stroma, the Descemet's membrane and the endothelial layer) from the anterior corneal layers, across the cornea up to the scleral spur. Another method is described by Zhu et al where a sheet of Descemet's membrane and endothelium, essentially free from stroma is obtained. Zhu et al., Cornea 25(6): 705-8 (2006); Melles et al., Cornea 23(3): 286-8 (2004).

Ultra thin grafts (i.e., <100 μm) can be prepared by double-pass automated microkeratomy where the cornea is sectioned twice. Sikder, Nordgren, and Moshifar, Am. J. Ophthalmol. 152(2): 202-208 (2011). By specifically choosing microkeratome heads based on the cornea thickness, a thin posterior bed can be obtained. The first resection removes the first 50 μm of anterior cap. The second pass resects the ultra-thin donor posterior bed. Hsu, Hereth, and Moshirfar, Clin. Ophthalmol. 6: 425-432 (2012). This method is referred to as the “double-pass” microkeratome technique and produces ultra-thin grafts, which are believed to produce improved visual outcomes. Hsu, Hereth, and Moshirfar, Clin. Ophthalmol. 6: 425-432 (2012); Neff, Biber, and Holland, Cornea 30(4): 388-391 (2011).

All the methods described above for resectioning corneas suffer from the difficulty in accurately achieving the specific thickness of the surgeon requested donor cornea posterior bed. The methods described herein permit the accurate and reproducible production of donor cornea posterior beds at specific thicknesses by modulating the pressure in the artificial anterior chamber during microtome operation. As such, the method produces donor cornea posterior beds in accordance with the surgeon's requested thickness and this can lead to improved visual outcomes for the patient.

### SUMMARY

The utilization of pressure optimization allows for increased accuracy in regards to the residual posterior bed. The increased accuracy allows for greater precision to meet the surgeon's preferences. The greater precision has led to requested residual posterior bed thickness of thinner grafts. The thinner grafts have, in turn, led to better visual acuity results in recipients.

One embodiment described herein is a method for increasing accuracy and precision in donor cornea posterior bed thickness during resectioning comprising selecting and maintaining a constant pressure under the cornea during resectioning.

In some aspects described herein, the constant pressure is about 11 PSI to about 20 PSI.

In other aspects described herein, the constant pressure is about 13 PSI to about 18 PSI

In other aspects described herein, the constant pressure is about 13.0 PSI; about 14.0 PSI; about 14.5 PSI; about 14.8 PSI; about 15.0 PSI; about 15.3 PSI; about 15.5 PSI; about 15.8 PSI; about 16.0 PSI; about 16.3 PSI; about 16.5 PSI; about 16.8 PSI; about 17.0 PSI; about 17.3 PSI; about 17.5 PSI; or about 18.0 PSI.

In other aspects described herein, the constant pressure is about 16 PSI.

In other aspects described herein, a microkeratome is used to perform the resection.

In other aspects described herein, the microkeratome has a nominal resection thickness range of about 50 μm to about 400 μm.

In other aspects described herein, the microkeratome has a nominal resection thickness range of about 200 μm to about 350 μm.

The method of claim 6, wherein the microkeratome has a nominal resection thickness of about 130 μm, about 200 μm, about 250 μm, about 300 μm, about 350 μm, or about 400 μm.

In other aspects described herein, a desired thickness of the posterior bed is from about 50 μm to about 250 μm.

In other aspects described herein, a desired thickness of the posterior bed is from about 80 μm to about 200 μm.

In other aspects described herein, a desired thickness of the posterior bed is about 80 μm, about 90 μm, about 100 μm, about 110 μm, about 120 μm, about 130 μm, about 140 μm, about 150 μm, about 160 μm, about 170 μm, about 180 μm, about 190 μm, or about 200 μm.

In other aspects described herein, a variance between a desired thickness of the posterior bed and a measured posterior bed thickness is less than 50 μm.

In other aspects described herein, a variance between a desired thickness of the posterior bed and a measured posterior bed thickness is less than 30 μm.

In other aspects described herein, a variance between a desired thickness of the posterior bed and a measured posterior bed thickness is less than 25 μm.

In other aspects described herein, an absolute value of a difference between a desired thickness of the posterior bed and a measured posterior bed thickness is from about 0 μm to about 70 μm.

In other aspects described herein, an absolute value of a difference between a desired thickness of the posterior bed and a measured posterior bed thickness is from about 0 μm to about 30 μm.

In other aspects described herein, an absolute value of a difference between a desired thickness of the posterior bed and a measured posterior bed thickness is from about 0 μm to about 15 μm.

In other aspects described herein, a difference in thickness between a desired thickness of the posterior bed and a measured posterior bed thickness is from about 0% to about 40%.

In other aspects described herein, a difference in thickness between a desired thickness of the posterior bed and a measured posterior bed thickness is from about 0% to about 25%.

In other aspects described herein, a difference in thickness between a desired thickness of the posterior bed and a measured posterior bed thickness is from about 0% to about 12%.

In other aspects described herein, the pressure is maintained by a pump, pressure syringe, IV bag, or other pressurizing means.

In other aspects described herein, the pressure is maintained by a pressure syringe.

In other aspects described herein, the keratomes are used in a multiple-pass procedure.

Another embodiment described herein is a method for preparing an accurate thickness of donor cornea posterior bed (i.e., endothelium and Descemet's membrane), suitable for transplantation, comprising: (a) selecting a desired thickness for a posterior bed; (b) measuring the cornea thickness of a donor cornea; (c) mounting the donor cornea in an apparatus connected to a pressurizing device; (d) increasing the pressure in the apparatus using the pressurizing device; (e) selecting at least one keratome apparatus based on the desired thickness of the posterior bed of (a) and the cornea thickness of (b); (f) using at least one of the selected keratomes to resect the donor cornea producing a lamellar graft; (g) measuring the thickness of the posterior bed produced in (f), and (h) wherein the difference between the desired thickness of the posterior bed of (a) and the thickness of the posterior bed produced in (f) is less than about 60 μm.

In some aspects described herein, an absolute value of the difference in thickness between the posterior bed in (a) and the thickness of the posterior bed produced in (f) is from about 0 μm to about 40 μm.

In other aspects described herein, an absolute value of the difference in thickness between the posterior bed in (a) and the thickness of the posterior bed produced in (f) is from about 0 μm to about 30 μm.

In other aspects described herein, an absolute value of the difference in thickness between the posterior bed in (a) and the thickness of the posterior bed produced in (f) is from about 0 μm to about 15 μm.

In other aspects described herein, an absolute value of the difference in thickness between the posterior bed in (a) and the thickness of the posterior bed produced in (f) is from about 0% to about 30%.

In other aspects described herein, the absolute value of the difference in thickness between the posterior bed in (a) and the thickness of the posterior bed produced in (f) is from about 0% to about 25%.

In other aspects described herein, the absolute value of the difference in thickness between the posterior bed in (a) and the thickness of the posterior bed produced in (f) is from about 0% to about 12%.

In other aspects described herein, the constant pressure is about 11 PSI to about 20 PSI.

In other aspects described herein, the constant pressure is about 13 PSI to about 18 PSI

In other aspects described herein, the constant pressure is about 13.0 PSI; about 14.0 PSI;

about 14.5 PSI; about 14.8 PSI; about 15.0 PSI; about 15.3 PSI; about 15.5 PSI; about 15.8 PSI; about 16.0 PSI; about 16.3 PSI; about 16.5 PSI; about 16.8 PSI; about 17.0 PSI; about 17.3 PSI; about 17.5 PSI; or about 18.0 PSI.

In other aspects described herein, the pressure is about 16 PSI.

In other aspects described herein, the keratome apparatus has a nominal resection thickness range of about 50 μm to about 400 μm.

In other aspects described herein, the keratome apparatus has a nominal resection thickness range of about 200 μm to about 350 μm.

In other aspects described herein, the desired thickness of the posterior bed is from about 50 μm to about 250 μm.

In other aspects described herein, the desired thickness of the posterior bed is from about 100 μm to about 150 μm.

In other aspects described herein, the pressurizing device comprises a pump, pressure syringe, IV bag, or other pressurizing means.

In other aspects described herein, the pressurizing device comprises a pressure syringe.

In other aspects described herein, the keratomes are used in a multiple-pass procedure.

In some aspects described herein, the method further comprising the steps: (i) trephining or punching the posterior bed produced in (f) to a specific circumferential size, resulting in a lenticule; and (j) storing the lenticule until needed for transplant.

In other aspects described herein, the circumferential size of the lenticule is from about 2 mm to about 10 mm.

In other aspects described herein, the circumferential size of the lenticule is from about 8 mm to about 10 mm.

Another embodiment described herein is donor cornea posterior bed produced by the method comprising: (a) selecting a desired thickness for a cornea posterior bed (i.e., endothelium and Descemet's membrane graft); (b) measuring the cornea thickness of a donor eye; (c) mounting the donor eye in an apparatus connected to a pressurizing device; (d) increasing the pressure in the apparatus using the pressurizing device; (e) selecting at least one microkeratome based on the desired thickness of the posterior bed of (a) and the cornea thickness of (b); (f) using at least one of the selected keratomes to resect the donor cornea producing a posterior bed; (g) measuring the thickness of the posterior bed produced in (f), and (h) wherein the difference between the measurement of (a) and the measurement of (f) is less than about 60 μm.

In some aspects used to produce the donor cornea posterior bed described herein, the pressurizing device comprises a pump, pressure syringe, IV bag, or other pressurizing means.

In some aspects used to produce the donor cornea posterior bed described herein, the pressurizing device comprises a pressure syringe.

In some aspects used to produce the donor cornea posterior bed described herein, the pressure in the apparatus is about 13.0 PSI; about 14.0 PSI; about 14.5 PSI; about 14.8 PSI; about 15.0 PSI; about 15.3 PSI; about 15.5 PSI; about 15.8 PSI; about 16.0 PSI; about 16.3 PSI; about 16.5 PSI; about 16.8 PSI; about 17.0 PSI; about 17.3 PSI; about 17.5 PSI; or about 18.0 PSI.

In some aspects used to produce the donor cornea posterior bed described herein, the pressure in the apparatus is about 16 PSI.

In some aspects used to produce the donor cornea posterior bed described herein, the method further comprises the steps: (i) trephining or punching the posterior produced in (f) to a specific circumferential size, resulting in a lenticule; and (j) storing the lenticule until needed for transplant.

### DETAILED DESCRIPTION

Several manufacturers of microkeratomes provide various incremental power driven cutting heads designed to remove specific thickness of anterior tissue. However, the cutting heads do not resect the specified thickness of posterior bed with great precision. In general, there is a variance between ±30 μm and ±100 μm between the expected posterior bed thickness and the actual thickness achieved in the resection procedure.

Typically, an artisan selects a particular microkeratome cutting head according to a nomogram chart. The donor cornea's specific pachometry (i.e., thickness) is compared to a table of predicted cutting thicknesses for various microkeratome cutting heads and the closet match is selected. As a non-limiting example, Moria has microkeratome heads with various nominal cut depths (Moria Inc., Doylestown, Pa.). A nominal 130 μm microkeratome head is predicted to remove 160 μm of anterior cornea tissue; a nominal 200 μm microkeratome head is predicted to remove 250 μm of anterior cornea tissue; a nominal 250 μm microkeratome head is predicted to remove 310 μm of anterior cornea tissue; a nominal 300 μm microkeratome head is predicted to remove 370 μm of anterior cornea tissue; a nominal 350 μm microkeratome head is predicted to remove 435 μm of anterior cornea tissue; and a nominal 400 μm microkeratome head is predicted to remove 500 μm of anterior cornea tissue. A nomogram having a line y=1.2514(x)+2.475, where y is the expected thickness of cornea resection and x is the nominal microkeratome head thickness, describes the relationship between the nominal head cut-depth and the expected amount of anterior cornea tissue removed. As the nominal depth increases, the variance in actual thickness increases.

Notwithstanding the apparent predictability in obtaining accurate posterior bed thickness depths, artisans with superior skill in the art typically, obtain variances of ±50 μm between nomogram-extrapolated expected thickness based on the nominal head depth and the actual thickness obtained. These discrepancies relate to the speed of the resection, thickness of the cornea, the age of the donor, the age of the tissue since it was harvested, the cornea cell morphology, edema, and the pressure applied to the cornea during resection. Therefore, a method of resecting corneas with greater accuracy and precision (i.e., less variability) in targeting specific surgeon requested posterior bed thickness is desirable. The method described herein permits greater accuracy and precision in such methods by modulating the pressure in the artificial anterior chamber during cornea resectioning.

### EXAMPLES

### Example 1

### Microscopic Evaluation of Donor Corneas

Specular and slit-lamp microscopies were performed on all donor corneas to verify endothelial cell health and density and to ensure no abnormal findings or evidence of previous refractive surgery was present. A review of the cornea was performed with the Visante OCT to ensure the cornea were of uniform thickness, curvature was acceptable, there was no evidence of refractive surgery, no abnormal findings were present, and obtain overall pachymetry. Corneas were rejected if the evaluations were unsatisfactory or would cause poor results if processed. The scleral rim was measured prior to processing and must meet specific measurements of a minimum of 16 mm overall diameter of the corneoscleral rim in order to be acceptable for further processing.

### Example 2

### Preparation for Corneal Resection

Two technicians performed the cornea resection procedure, one who was sterile, and the other who was not sterile. The following items were sterilized and placed into a sterile field by the non-sterile technician:
microkeratome blade (Moria);
specimen cup;
gauze pads;
tubing cover;
Optisol GS;
10 mL balance salt solution (BSS);
ocular sponges;
10 mL (cc) syringe;
skin marker (if needed); and
cornea viewing chamber (if requested).

If a cornea viewing chamber was used, then the sterile technician moved the viewing chamber near the edge of the sterile field and removed the lid. The non-sterile technician then aseptically decanted the entire contents of the Optisol GS (Bausch & Lomb, Irvine, Calif.) vial into the chamber. The sterile technician replaced the lid and set the chamber away from the processing area of the sterile field.

The non-sterile technician then passed the pachymeter probe to the sterile technician. The non-sterile technician verified the Identity of the subject cornea with the sterile technician and brought it near the tissue-processing surface with the surgeon's request form. Both the tissue ID number and the surgeon's request were verified by both technicians simultaneously to ensure correctness.

The non-sterile technician ensured that the cornea was in the vial with the endothelium side up, uncapped the cornea vial near the tissue-processing surface, and then poured the entire contents of the vial into the sterile specimen cup that was being held by the sterile technician near the edge of the sterile field. The sterile technician set the sterile specimen cup containing the donor cornea on the sterile field in a safe area to prevent knocking over the specimen cup and cornea.

The sterile technician drew 10 mL of media (Optisol GS) from the specimen cup containing the subject cornea with the sterile 10 mL syringe. The syringe with Optisol GS was then attached to the middle port of a 3-way stopcock connector that was attached to a pressure syringe (B. Braun Inflation Device, No. 622510; B. Braun, Bethlehem Pa.) on one port and an artificial anterior chamber (Moria) on the other port. The stopcock was opened to the syringe port and the artificial anterior chamber was flushed with several milliliters of Optisol GS. The Optisol was blotted with a laboratory sponge to prevent the fluid from spilling onto the sterile field.

The donor cornea was prepared, if necessary, such as trimming the scleral rim, removing uveal tissue, removing conjunctiva, etc.

The cornea was then positioned on the base of the artificial anterior chamber at its lowest position by turning the graft tightening ring counterclockwise. Optisol GS was added to the artificial anterior chamber to create the highest possible meniscus on top of the chamber. The cornea was then picked up using forceps and placed on the artificial anterior chamber at about a 45-degree angle while applying light pressure on the 10 cc syringe to dispense enough media into the chamber and displace any air bubbles while the cornea was lowered onto the chamber. The cornea was centered and inspected to ensure that an irregular rim did not cause the cornea to be off center. The cup cover was positioned on the base locking ring and the crenels of the cup cover were inserted into the base locking ring's bayonet. The cup cover was turned about 15 degrees clockwise to lock the cup cover in place. The piston with the cornea was moved upward by rotating the graft-tightening ring clockwise. The piston was moved upward until the cornea contacted the upper lumen of the cup cover and then firmly tightened by hand to ensure adequate sealing of the system and to secure the cornea during the microkeratome cut.

The cornea epithelium was removed as needed with an ocular sponge and the balanced salt solution.

The adjusting ring was then positioned on top of the cup cover and the guide ring with the microkeratome track was positioned on the top of the cup cover. The guide ring was locked in place with a set screw. The guide ring and adjusting ring were set to expose the maximum diameter of cornea by turning the adjusting ring clockwise as far as possible. Precut cornea markings were placed on the cornea at this time, unless otherwise noted by the surgeon.

Air pressure was established in the pressure syringe using the following method. The right portal of the 3-way stopcock connector was attached to tubing connected to the artificial anterior chamber. The green tightening wing on the B. Braun pressure syringe was loosened, the plunger was pulled back all the way back to the end, and then the green tightening wing was locked in place. The B. Braun pressure syringe was then attached to tubing connected to the left portal of the 3-way stopcock connector. The B. Braun pressure syringe side of the 3-way stopcock connector portal was turned to the “OFF” position or closed and the B. Braun screw plunger was advanced until it reached the 8-14 increment range on the pressure syringe. Typically, a pressure setting of about 11 was used. This corresponds to a pressure of 16 PSI or 1.09 atm.

This procedure increases pressure in the artificial anterior chamber to a level necessary for accurately resecting the cornea. The pressure enables correct applanation of the cornea during the microkeratome operation.

TABLE 1
Pressure of the Artificial Anterior Chamber with B. Braun Pressure
Syringe

| | Pounds per Square Inch | Atmospheres |
|---|---|---|
| B. Braun Syringe Setting | (PSI) | (ATM) |
| 8 | 19 | 1.29 |
| 9 | 18 | 1.22 |
| 10 | 17 | 1.16 |
| 11 | 16 | 1.09 |
| 12 | 15 | 1.02 |
| 13 | 14 | 0.95 |
| 14 | 13 | 0.88 |

### Example 3

### Corneal Resection

A minimum of three pachymetry measurements were taken from the central cornea and were used to ensure accuracy of measurements. The average of the three measurements was then calculated and recorded by the non-sterile technician. The goal was to cut an anterior resection that leaves 100 to 200 μm of posterior cornea remaining. A microtome head was then selected. The head choice size was determined by a combination of the thickness of the cornea, the surgeon's requested thickness of the remaining posterior bed, and the estimated average removed by the appropriate head. A Moria microkeratome head marked 400 μm typically removes an average of 500 μm of anterior cornea material. A Moria microkeratome head marked 350 μm typically removes an average of 435 μm of anterior cornea material. A Moria microkeratome head marked 300 μm typically removes an average of 370 μm of anterior cornea material. A Moria microkeratome head marked 250 μm typically removes an average of 310 μm of anterior cornea material. A Moria microkeratome head marked 200 μm typically removes an average of 250 μm of anterior cornea material.

If a surgeon requested a specific head, then this head was used. If the precut cornea measurements and the use of this head would cause poor results, the surgeon was consulted to make a decision prior to processing.

After the appropriate microtome head was selected, the blade was inserted. The Moria microkeratome head was then attached to the Moria turbine while taking care to hold the blade in place (the head may also be loosely set on the turbine and the blade inserted prior to tightening). Once the head and blade were in place, the microtome control foot pedal was depressed to ensure that the blade reciprocates correctly before the head was placed into the track on the guide ring.

The 3-way stopcock connector attached to the syringe containing the Optisol GS (i.e., the media port) was then closed and the port to the B. Braun pressure syringe (i.e., the air port) was opened. A few drops of BSS were placed near the starting position of the track on the guide ring to ensure smooth movement of the microkeratome. The microkeratome was picked up and re-tested for function by temporarily depressing the turbine pedal. The microkeratome head was fed into the track on the guide ring and checked for free mobility. The cornea was resected by depressing and holding turbine pedal and moving the microkeratome continuously through the cornea without stopping, while even downward pressure was applied to the microkeratome head. After the resection was completed, the turbine pedal was released. Then, the air port (B. Braun pressure syringe) on the stopcock was closed and the media port was opened. The sterile technician then removed the cut portion of the cornea from the microkeratome blade area with forceps, taking care to maintain the correct orientation and not turn the cap inside out.

### Example 4

### Post-Resection Procedure

The resected cornea cap was placed on a sterile metric ruler and the diameter was measured (in millimeters) and documented. The resected cornea cap was then placed back onto the cornea, taking care to maintain the correct, original biological orientation. The edges of the cornea/resection interface were dried with ocular sponges until it appeared that the resected cap would remain in place.

Post-cut cornea markings were made on the cornea with a sterile skin marker, unless otherwise noted by the surgeon. The guide ring and adjusting ring were removed from the cup cover. The artificial anterior chamber piston was slowly lowered by turning the graft tightening ring counter-clockwise. The pressure was maintained in the artificial anterior chamber by adding cornea storage media from the syringe as the artificial anterior chamber piston was lowered. Maintaining the pressure was important so that the cornea does not collapse. The seal of the cornea/chamber junction was broken by dripping BSS on the cornea/ring junction and by gently sweeping the cornea with forceps at the juncture. The cornea and resected cap were removed from the artificial anterior chamber and placed in a new, labeled vial of media (or viewing chamber with media if requested by the surgeon).

If a cornea viewing chamber was used, the sterile technician removed the lid and placed it on the sterile field with the inside facing upwards. The sterile technician then cut a small “V”-shaped notch in the rim to allow the Optisol to reach the endothelium. The cornea was then placed endothelium-side up in the chamber, the lid was replaced and tightened, and the chamber was handed to the non-sterile technician.

Specular Microscopy was then performed on the resected cornea/cornea cap to verify endothelial cell health and cell density. Slit Lamp Microscopy was also performed to ensure the resection was centered, the posterior bed was of uniform thickness, and to ensure that no other abnormal findings were present. Optical Coherence Tomography was performed to ensure the posterior bed was of uniform thickness and that no other abnormal findings were present. The vials were then labeled and packaged for shipment or stored at 4° C.

Data from about 200 DSAEK procedures are presented in Table 2, below. The shaded data at the bottom of the table were procedures where the thicknesses of the cornea posterior bed graft were greater than the surgeon-requested posterior bed thickness that would be expected based on the microkeratome head expected thickness, and thus were negative results (i.e., −).

TABLE 2
Posterior Bed Outcomes Achieved with Artificial Anterior Chamber Pressure Optimization

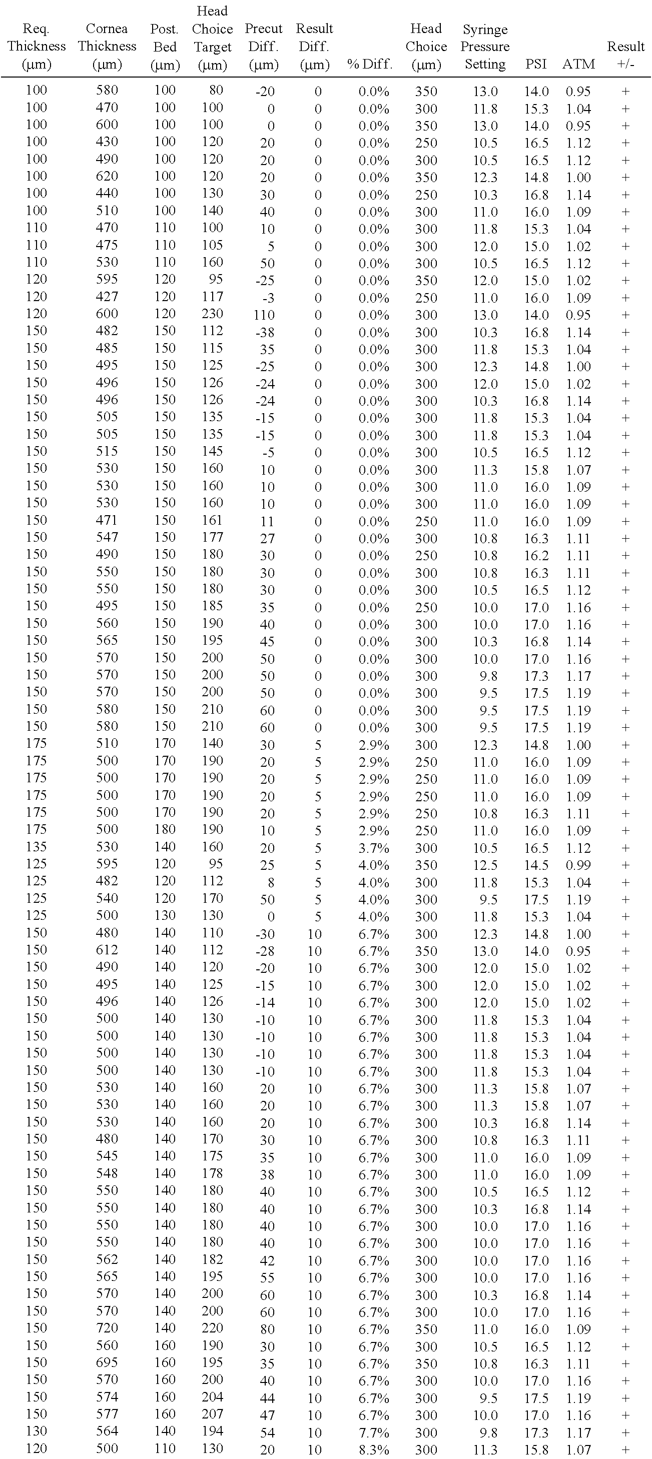

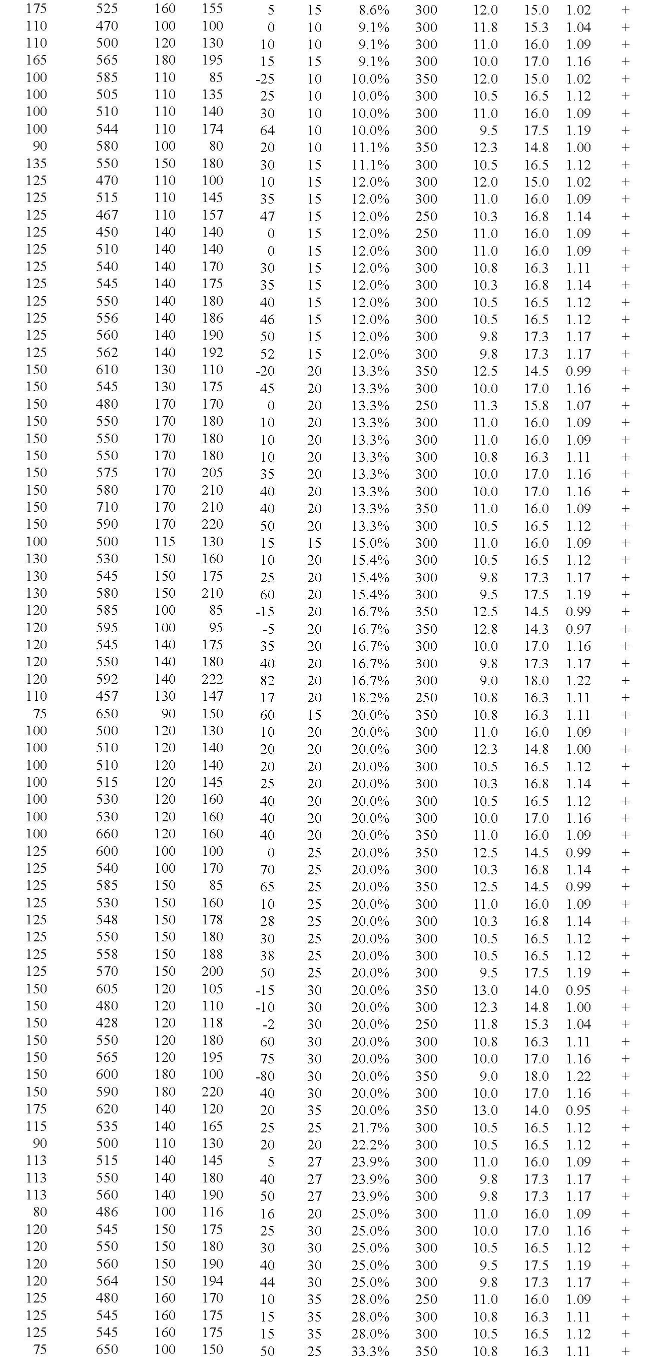

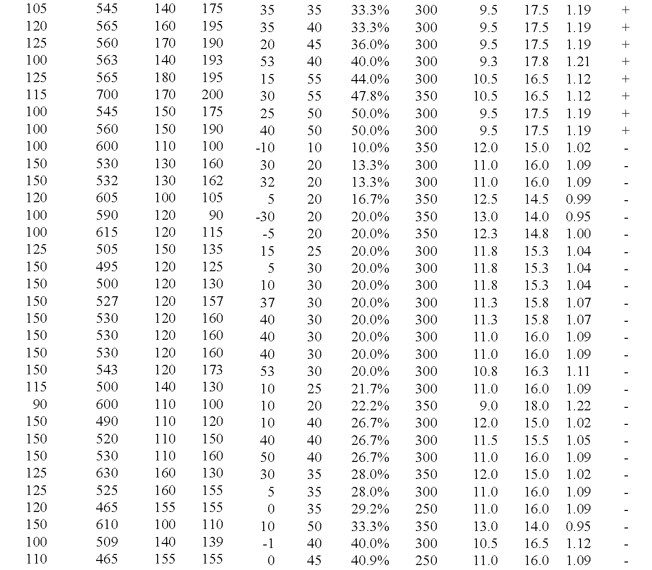

### Example 5

### Outcomes

The corneal resection procedure described above where the pressure in the artificial anterior chamber is maintained at 13-19 PSI (0.88-1.29 atm) produces corneal posterior bed thicknesses closer to the surgeon-requested thickness. For the data displayed above, using pressures from 14-18 PSI (0.95-1.22 atm), 167 DSAEK procedures produced outcomes that were closer to the surgeon-requested posterior bed thickness than one would expect based on the microkeratome head expected thickness alone. This is an 86.98% success rate (i.e., 167 out of 192 procedures). Of the successful DSAEK procedures, the mean pressure used was 16.2±0.07 PSI (1.10 atm); the median was 16.25 PSI (1.11 atm), and the mode was 16 PSI (1.09 atm). The procedures that differed more than the microkeratome head expected thickness (i.e., were thicker or thinner than the surgeon requested) had a mean pressure of 15.6 PSI (1.06 atm), slightly lower than the successful procedures.

Approximately 92% of the 167 successful cornea resection procedures had posterior bed thickness within 30 μm of what the surgeon had requested. Approximately 60% of the successful procedures had posterior bed thicknesses within 15 μm of the graft thickness requested by the surgeon. Of the successful procedures, 22.4% exactly met the surgeon's requested thickness. Approximately 96% of the cornea resection procedures had 40% or less difference between actual posterior bed thickness and the surgeon's requested graft thickness. Approximately 90% of the cornea resection procedures had 25% or less difference between actual posterior bed thickness and the surgeon's requested graft thickness. Approximately 46% of the cornea resection procedures had 12% or less difference between actual posterior bed thickness and the surgeon's requested graft thickness. Approximately 22% of the cornea resection procedures met the surgeon's requested posterior bed graft thickness.

The manipulation of the pressure in the artificial anterior chamber has not previously been used to prepare more accurate and precise thicknesses of corneal posterior bed grafts. Methods described in prior art suffer from the difficulty in accurately achieving the specific thickness of the surgeon-requested donor cornea posterior bed. Artisans with superior skill in the art typically obtain variances of ±50 μm between nomogram-extrapolated expected thickness based on the nominal head depth and the actual thickness obtained. These discrepancies relate to the speed of the resection, thickness of the cornea, the age of the donor, the age of the tissue since it was harvested, the cornea cell morphology, edema, and the pressure applied to the cornea during resection. Therefore, a method of resecting corneas with greater accuracy and precision (i.e., less variability) in targeting specific surgeon requested posterior bed thickness is desirable. The methods described herein permit the accurate and reproducible production of donor cornea posterior beds at specific thicknesses by modulating the pressure in the artificial anterior chamber during microtome operation. As such, the method produces donor cornea posterior beds in accordance with the surgeon's requested thickness and this can lead to improved visual outcomes for the patient.

While the foregoing disclosure has been described in some detail for purposes of clarity and understanding, it will be appreciated by one skilled in the art from a reading of this disclosure that various changes in form and detail can be made without departing from the true scope of the disclosure and appended claims. All patents and publications cited herein are entirely incorporated herein by reference.

What is claimed is:

1. A method for increasing accuracy and precision in donor cornea posterior bed thickness during resectioning comprising selecting and maintaining a constant pressure under the cornea during resectioning.

2. The method of claim 1, wherein the constant pressure is about 11 PSI to about 20 PSI.

3. (canceled)

4. (canceled)

5. (canceled)

6. The method of claim 1, wherein a microkeratome is used to perform the resection.

7. The method of claim 6, wherein the microkeratome has a nominal resection thickness range of about 50 μm to about 400 μm.

8. (canceled)

9. (canceled)

10. The method of claim 1, wherein a desired thickness of the posterior bed is from about 50 μm to about 250 μm.

11. (canceled)

12. (canceled)

13. The method of claim 1, wherein a variance between a desired thickness of the posterior bed and a measured posterior bed thickness is less than 50 μm.

14. (canceled)

15. (canceled)

16. The method of claim 1, wherein an absolute value of a difference between a desired thickness of the posterior bed and a measured posterior bed thickness is from about 0 μm to about 70 μm.

17. (canceled)

18. (canceled)

19. The method of claim 1, wherein a difference in thickness between a desired thickness of the posterior bed and a measured posterior bed thickness is from about 0% to about 40%.

20. (canceled)

21. (canceled)

22. The method of claim 1, wherein the pressure is maintained by a pump, pressure syringe, IV bag, or other pressurizing means.

23. (canceled)

24. The method of claim 1, wherein the keratomes are used in a multiple-pass procedure.

25. A method for preparing an accurate thickness of donor cornea posterior bed, suitable for transplantation, comprising:
(a) selecting a desired thickness for a posterior bed;
(b) measuring the cornea thickness of a donor cornea;
(c) mounting the donor cornea in an apparatus connected to a pressurizing device;
(d) increasing the pressure in the apparatus using the pressurizing device;
(e) selecting at least one keratome apparatus based on the desired thickness of the posterior bed of (a) and the cornea thickness of (b);
(f) using at least one of the selected keratomes to resect the donor cornea producing a lamellar graft;
(g) measuring the thickness of the posterior bed produced in (f), and
(h) wherein the difference between the desired thickness of the posterior bed of (a) and the thickness of the posterior bed produced in (f) is less than about 60 μm.

26. The method of claim 25, wherein an absolute value of the difference in thickness between the posterior bed in (a) and the thickness of the posterior bed produced in (f) is from about 0 μm to about 40 μm.

27. (canceled)

28. (canceled)

29. The method of claim 25, wherein an absolute value of the difference in thickness between the posterior bed in (a) and the thickness of the posterior bed produced in (f) is from about 0% to about 30%.

30. (canceled)

31. (canceled)

32. The method of claim 25, wherein the constant pressure is about 11 PSI to about 20 PSI.

33. (canceled)

34. (canceled)

35. (canceled)

36. The method of claim 25, wherein the keratome apparatus has a nominal resection thickness range of about 50 μm to about 400 μm.

37. (canceled)

38. (canceled)

39. (canceled)

40. The method of claim 25, wherein the pressurizing device comprises a pump, pressure syringe, IV bag, or other pressurizing means.

41. (canceled)

42. The method of claim 25, wherein the keratomes are used in a multiple pass procedure.

43. The method of claim 25, further comprising the steps:
(i) trephining or punching the posterior bed produced in (f) to a specific circumferential size, resulting in a lenticule; and
(j) storing the lenticule until needed for transplant.

44. The method of claim 43, wherein the circumferential size of the lenticule is from about 2 mm to about 10 mm.

45. (canceled)

46. A donor cornea posterior bed produced by the method comprising:
(a) selecting a desired thickness for a cornea posterior bed (i.e., endothelium and Descemet's membrane graft);
(b) measuring the cornea thickness of a donor eye;
(c) mounting the donor eye in an apparatus connected to a pressurizing device;
(d) increasing the pressure in the apparatus using the pressurizing device;
(e) selecting at least one microkeratome based on the desired thickness of the posterior bed of (a) and the cornea thickness of (b);
(f) using at least one of the selected keratomes to resect the donor cornea producing a posterior bed;
(g) measuring the thickness of the posterior bed produced in (f), and
(h) wherein the difference between the measurement of (a) and the measurement of (f) is less than about 60 μm.

47. (canceled)

48. (canceled)

49. (canceled)

50. (canceled)

51. (canceled)
